(19) United States
(12) Patent Application Publication
(10) Pub. No.: US 2026/0325765 A1
(43) Pub. Date: Oct. 22, 2026

(54) PRESSURE CONTROL SYSTEM AND PRESSURE CONTROL METHOD

(71) Applicants: Haruhiro INOUE, Tokyo (JP); OLYMPUS MEDICAL SYSTEMS CORP., Tokyo (JP)

(72) Inventors: Haruhiro INOUE, Tokyo (JP); Masahiko OKAMURA, Tokyo (JP); Yuma KASUYA, Tokyo (JP)

(73) Assignees: Haruhiro INOUE, Tokyo (JP); OLYMPUS MEDICAL SYSTEMS CORP., Tokyo (JP)

(21) Appl. No.: 19/696,824

(22) Filed: Jun. 3, 2026

Related U.S. Application Data
(63) Continuation of application No. PCT/JP2023/043288, filed on Dec. 4, 2023.

(51) Int. Cl. A61M 13/00; A61B 1/00; A61B 1/015; A61B 1/273

(52) U.S. Cl. CPC A61M 13/003; A61B 1/00119; A61B 1/015; A61B 1/2736; A61M 2205/3344; A61M 2205/75

(57) ABSTRACT

A pressure control system includes a first tube configured to communicate with a lumen within a body of an examinee, a pressure sensor disposed at the first tube and configured to measure a pressure of a gas, and a first valve disposed at the first tube and configured to vent the gas to an atmosphere. The pressure control system further includes a second tube configured to communicate with the lumen and supply the gas from a gas supply source to the lumen, a second valve disposed at the second tube and configured to vent the gas to the atmosphere, and processing circuitry. The processing circuitry is configured to control the first valve to open based on the pressure, and control the second valve to open when a decrease rate of the pressure after controlling the first valve to open is equal to or less than a predetermined rate.

### CROSS REFERENCE TO RELATED APPLICATION

This application is a continuation of International Application No. PCT/JP 2023/043288, filed on Dec. 4, 2023, the entire contents of which are incorporated herein by reference.

### FIELD

Aspects of the present disclosure relate to a pressure control system and a pressure control method for controlling a pressure in a lumen within a body of an examinee.

### BACKGROUND

Gastroesophageal reflux disease (hereinafter referred to as “GERD”) is a functional disorder where, for example, the function of the gastric cardia deteriorates, causing gastric fluid to reflux to the esophagus. As methods for diagnosing whether an examinee has GERD (hereinafter referred to as “GERD diagnostic methods”), techniques such as 24-hour esophageal pH monitoring and 24-hour esophageal impedance pH monitoring are used.

To mitigate the burden on the examinee in GERD diagnostic methods, a method using an endoscope to observe the relaxation state of the lower esophageal sphincter (LES) at the gastric cardia is increasingly being employed.

International Publication No. 2021/166127 discloses an endoscope system that diagnoses GERD based on pressure change patterns by measuring the pressure within a lumen of a subject while supplying gas into the lumen.

Esophageal achalasia, where an abnormality occurring in the function of the lower esophageal sphincter causes difficulty in passing food from the esophagus to the stomach, opposite to GERD, is also one of the esophageal functional disorders.

### SUMMARY

According to aspects of the present disclosure, a pressure control system is provided, which includes a first tube, a pressure sensor, a first valve, a second tube, a second valve, and processing circuitry. The first tube is configured to communicate with a lumen within a body of an examinee. The pressure sensor is disposed at the first tube and configured to measure a pressure of a gas. The first valve is disposed at the first tube and configured to vent the gas to an atmosphere. The second tube is configured to communicate with the lumen and supply the gas from a gas supply source to the lumen. The second valve is disposed at the second tube and configured to vent the gas to the atmosphere. The processing circuitry is configured to control the first valve to open based on the pressure. The processing circuitry is further configured to control the second valve to open when a decrease rate of the pressure after controlling the first valve to open is equal to or less than a predetermined rate.

According to aspects of the present disclosure, further provided is a pressure control system that includes a first tube, a pressure sensor, a first valve, a second tube, a second valve, and processing circuitry. The first tube is configured to communicate with a lumen within a body of an examinee. The pressure sensor is disposed at the first tube and configured to measure a pressure of a gas. The first valve is disposed at the first tube and configured to vent the gas to an atmosphere. The second tube is configured to communicate with the lumen and supply the gas from a gas supply source to the lumen. The second valve is disposed at the second tube and configured to vent the gas to the atmosphere. The processing circuitry is configured to control the second valve to open when the pressure is equal to or greater than a first pressure. The processing circuitry is further configured to control the first valve to open when a decrease rate of the pressure after controlling the second valve to open is equal to or less than a predetermined rate.

According to aspects of the present disclosure, further provided is a pressure control method implemented by processing circuitry operatively coupled to a pressure control system. The pressure control system includes a first tube, a pressure sensor, a first valve, a second tube, and a second valve. The pressure control method includes controlling the first valve to open based on a pressure of a gas measured by the pressure sensor disposed at the first tube. The pressure control method further includes controlling the second valve to open when a decrease rate of the pressure after controlling the first valve to open is equal to or less than a predetermined rate. The first tube is configured to communicate with a lumen within a body of an examinee. The first valve is disposed at the first tube and configured to vent the gas to an atmosphere. The second tube is configured to communicate with the lumen and supply the gas from a gas supply source to the lumen. The second valve is disposed at the second tube and configured to vent the gas to the atmosphere.

### BRIEF DESCRIPTION OF DRAWINGS

FIG. 1 is a general configuration diagram of a pressure control system including a pressure control apparatus according to a first embodiment of the present disclosure;

FIG. 2 is a configuration diagram of the pressure control system including the pressure control apparatus according to the first embodiment of the present disclosure;

FIG. 3 is a flowchart of a pressure control method according to the first embodiment of the present disclosure;

FIG. 4 is a graph showing pressure changes in a lumen of an examinee with GERD according to the embodiment of the present disclosure;

FIG. 5 is a graph showing pressure changes in a lumen of a healthy subject according to the embodiment of the present disclosure;

FIG. 6 is a graph showing pressure changes in a lumen of a patient with achalasia according to a conventional control system;

FIG. 7 is a graph showing pressure changes in a lumen of a patient with achalasia according to the embodiment of the present disclosure;

FIG. 8 is a configuration diagram of a pressure control system including a pressure control apparatus according to a second embodiment of the present disclosure; and

FIG. 9 is a flowchart of a pressure control method according to the second embodiment of the present disclosure.

### DESCRIPTION OF EMBODIMENTS

Hereinafter, embodiments will be described with reference to the drawings. It should be noted that the drawings based on the embodiments are schematic. The relationship between the thickness and width of each part and the ratio of the thickness of each part in the drawings differ from the actual ones. The drawings also contain parts in which dimensional relationships and ratios are different from each other. Illustration of some components and their reference numerals are omitted.

### First Embodiment

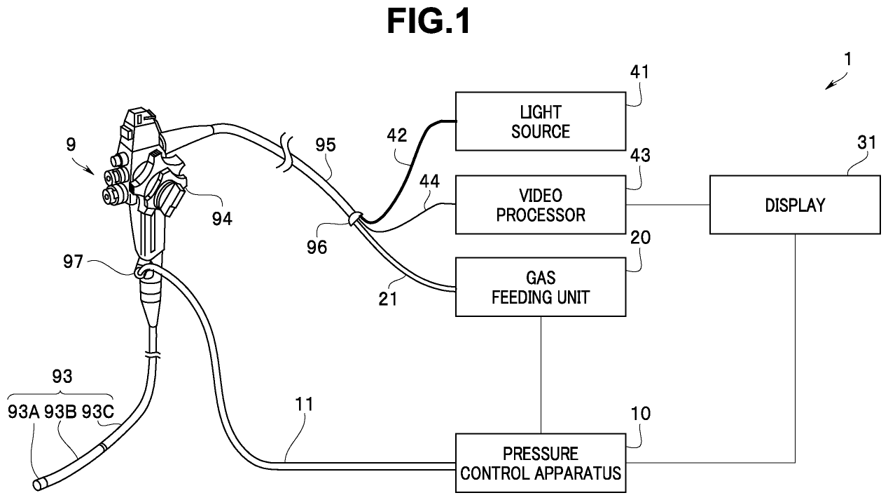

A pressure control system 1 according to the present embodiment shown in FIG. 1 includes an endoscope 9, a pressure control apparatus 10, a gas feeding unit 20, a display 31, a video processor 43, and a light source 41. The endoscope 9, the pressure control apparatus 10, the gas feeding unit 20, the display 31, the video processor 43, and the light source 41 are hardware.

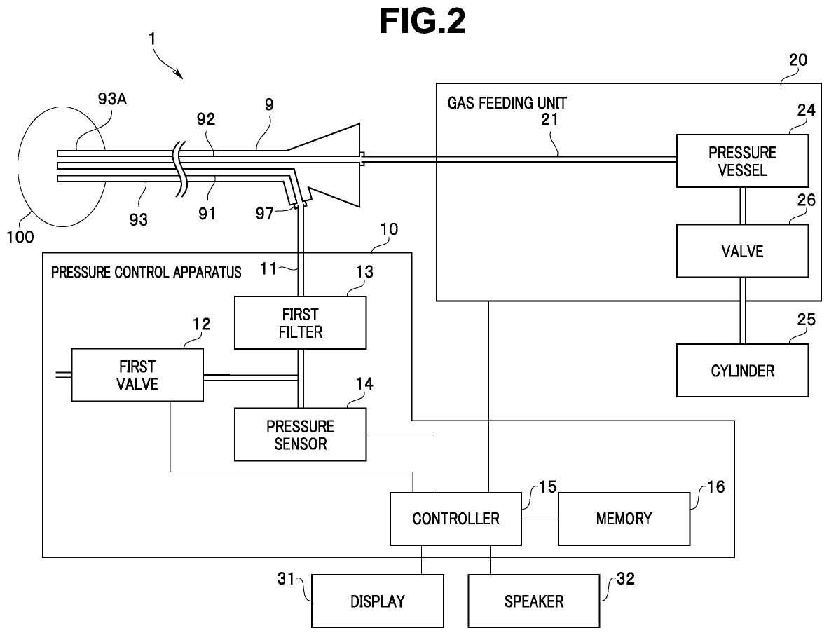

The endoscope 9 includes an elongated insertion portion 93 to be inserted into a subject, an operation portion 94 provided at a proximal end of the insertion portion 93, a universal cord 95 extending from the operation portion 94, and a connector 96. The operation portion 94 includes a bending operation knob and a plurality of buttons for operating endoscope functions. The insertion portion 93 of the endoscope 9 includes, in order from a distal end side, a distal end portion 93A, a bendable bending portion 93B provided at a proximal end of the distal end portion 93A, and an elongated flexible pipe 93C provided at a proximal end of the bending portion 93B. Although not shown, the distal end portion 93A is provided with an image pickup unit, an illumination optical system, and an opening of a forceps channel 91 (FIG. 2). The forceps channel 91 is a tube passing through the insertion portion 93 from an insertion port 97 of the operation portion 94 to the opening of the distal end portion 93A.

The connector 96 connects to an optical fiber bundle 42 of the light source 41, a cable 44 of the video processor 43, and a second tube (gas feeding tube) 21 of the gas feeding unit 20. Illumination light generated by the light source 41 is guided via the optical fiber bundle 42 to the illumination optical system of the distal end portion 93A. The gas feeding unit 20 ejects, in response to a button operation of the operation portion 94, a fluid (gas or liquid) from the opening of the distal end portion 93A via the gas/liquid feeding pipe 92 (FIG. 2) passing through the universal cord 95, the operation portion 94, and the insertion portion 93. That is, the gas feeding unit 20 is a gas/liquid feeding unit capable of supplying not only gas but also liquid, but in the description of the present disclosure, the fluid used is only gas. Gas/liquid feeding is switched by a button operation of the operation portion 94. The second tube 21 may be connected to the operation portion 94.

The video processor 43 processes an image pickup signal from the image pickup unit of the distal end portion 93A and displays an endoscopic image on the display 31.

The pressure control system 1 of the present embodiment is configured by adding the pressure control apparatus 10 to an endoscope system mentioned above.

As shown in FIG. 2, the pressure control apparatus 10 includes a first tube 11, a first valve 12, a first filter 13, a pressure sensor 14, a controller 15, and a memory 16. The first tube 11, the first valve 12, the first filter 13, the pressure sensor 14, the controller 15, and the memory 16 are hardware.

The first tube 11 is connected to the insertion port 97 of the forceps channel 91 of the endoscope 9. When the distal end portion 93A of the insertion portion 93 of the endoscope 9 is inserted into the stomach 100, which is a lumen of the subject, the first tube 11 communicates with the interior of the stomach 100 via the forceps channel 91. The first valve 12 and the first filter 13 are disposed at the first tube 11, and the pressure sensor 14 is disposed at the first tube 11.

The first valve 12 is a relief valve that vents gas within the first tube 11 to the atmosphere. The pressure sensor 14 measures a pressure of the gas within the first tube 11. Since the first tube 11 communicates with the stomach 100, the first valve 12 vents gas within the stomach 100 to the atmosphere, and the pressure sensor 14 measures a pressure of the gas within the stomach 100. The first filter 13 for inhibiting contamination of the pressure sensor 14 is disposed at a position closer to the stomach 100 than the pressure sensor 14.

The controller 15 may be implemented by processing circuitry including one or more processors and non-transitory computer-readable storage media. For example, the controller 15 including a central processing unit (CPU) performs control of the entirety of the pressure control system 1. In this case, for instance, the controller 15 may be configured to take overall control of the pressure control system 1 by the CPU executing one or more programs stored in non-transitory computer-readable storage media. The memory 16 stores setting conditions and the like for the pressure control system 1.

The gas feeding unit 20 includes the second tube 21, a pressure vessel 24, and a valve 26. The second tube 21 is connected to the gas/liquid feeding pipe 92 of the endoscope 9. When the distal end portion 93A of the insertion portion 93 of the endoscope 9 is inserted into the stomach 100, which is a lumen of the subject, the second tube 21 communicates with the interior of the stomach 100 via the gas/liquid feeding pipe 92.

Gas, such as carbon dioxide gas, supplied from a cylinder 25, which is a gas supply source, is decreased in pressure by a regulator (not shown) and fed to the second tube 21 via the pressure vessel 24.

The pressure control system 1 diagnoses GERD and achalasia by measuring a pressure (internal pressure) of gas within the stomach 100, while feeding gas from the gas feeding unit 20 to the stomach 100 of the subject via the endoscope 9. Pressure data is displayed on the display 31 in a time series. The display 31 displays the pressure data together with an endoscopic image, but may be a display dedicated to pressure data. A speaker 32 notifies a user audibly, for example, of an abnormality occurrence.

A tolerable pressure of the gas within the stomach 100 is set to a first pressure P1 at which the examinee experiences extreme discomfort. As will be mentioned later, when the pressure reaches the first pressure P1, the controller 15 controls the valve 26 to close so that the gas feeding unit 20 stops supplying gas. However, the pressure of the gas within the second tube 21 and the pressure of the gas within the pressure vessel 24 are higher than the first pressure P1, which is the pressure of the gas within the stomach 100. For this reason, even after the valve 26 is controlled to close, gas may be fed to the stomach 100, causing the internal pressure to exceed the first pressure P1.

In the pressure control system 1, when the internal pressure reaches the first pressure P1, not only the valve 26 is controlled to close, but also the first valve 12 is controlled to open. Since there is no risk that the internal pressure significantly exceeds the first pressure P1, the pressure control system 1 can mitigate the burden on the examinee.

### Pressure Control Method

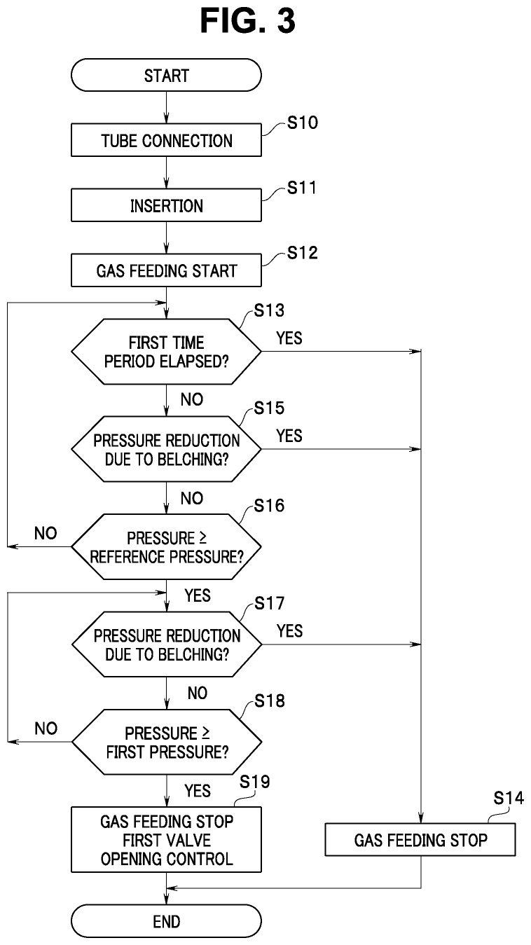

A pressure control method by the pressure control system 1 will be described along the flowchart of FIG. 3.

### <Step S10>Tube Connection

The first tube 11 is connected to the insertion port 97 of the forceps channel 91 of the endoscope 9. The second tube 21 is connected to the gas/liquid feeding pipe 92 of the endoscope 9.

### <step S11>Insertion

The distal end portion 93A of the insertion portion 93 of the endoscope 9 is inserted into the interior of the stomach 100 of the subject. It should be noted that observation of the cardia may be performed using the image pickup unit disposed at the distal end portion 93A.

### <Step S12>Gas Feeding Start

The controller 15 controls the valve 26 of the gas feeding unit 20 to feed gas into the stomach 100. The valve 26 is, for example, a type of solenoid valve, and includes a regulating valve using an electromagnetic coil as a drive section. When a current is caused to flow through the electromagnetic coil, a magnetic force is generated, attracting the plunger to move the valve. The magnitude of the current caused to flow through the electromagnetic coil controls an opening degree of the valve, adjusting a flow rate of gas to a predetermined value.

### <Step S13>First Time Period Elapsed?

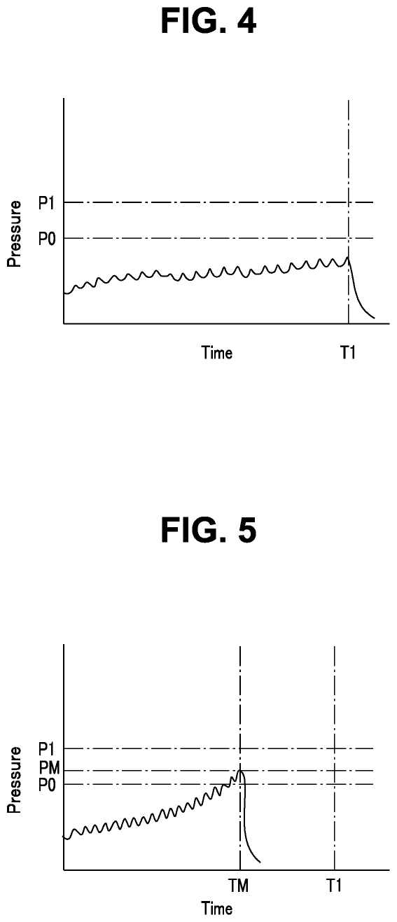

FIG. 4 shows changes in gastric internal pressure with respect to gas feeding time period in a patient with GERD with deteriorated function of the gastric cardia. It should be noted that gastric internal pressure reflects respiratory variation, and rises during inspiration relative to expiration. For this reason, the measured pressure repeatedly increases and decreases according to the respiratory cycle.

In a patient with GERD, even when gas is fed into the stomach 100, the gas continues to leak from the cardia. For this reason, even after a predetermined first time period T1 has elapsed since gas feeding starts, a pressure P does not reach a reference pressure P0. The reference pressure P0 is a pressure for diagnosing GERD. The first time period T1 and the reference pressure P0 are set as appropriate; for example, T1 is 5 minutes and P0 is 16 mmHg.

When the first time period T1 elapses from the start of gas feeding (S13, YES), the process proceeds to step S14.

### <step S14>Gas Feeding Stop

The gas feeding from the gas feeding unit 20 is stopped under control of the controller 15, and the process ends. It should be noted that, when stopping gas feeding, the controller 15 may control the gas feeding unit 20 to lower the flow rate of gas feeding to a predetermined flow rate and then completely stop the gas feeding.

### <Step S15>Pressure Reduction Due to Belching?

FIG. 5 shows changes in gastric internal pressure with respect to gas feeding time period in a healthy subject. The pressure gradually increases, but at a pressure PM (time TM), gas within the stomach 100 is expelled from the mouth by belching, causing the pressure to reduce sharply.

If the controller 15 detects that the pressure has decreased sharply (S15: YES), the process proceeds to step S14.

### <Step S16>Pressure≥Reference Pressure?

The processes from step S13 are repeated until the pressure is equal to or greater than the reference pressure P0.

### <Step S17>Pressure Reduction Due to Belching?

If the pressure PM during a pressure reduction due to belching exceeds the reference pressure P0, the examinee is diagnosed as not having GERD. For a healthy subject, a pressure PM at which belching occurs is, for example, greater than 18 mmHg and less than 25 mmHg.

### <step S18>Pressure≥First Pressure?

In a patient with achalasia, due to an abnormality occurring in the function of the lower esophageal sphincter, the pressure rises to exceed the pressure PM at which belching occurs in a healthy subject. For example, the examinee is diagnosed as having achalasia when the pressure PM at which belching occurs exceeds 20 mmHg. To diagnose the severity of achalasia, gas feeding is further continued until the pressure is equal to or greater than the first pressure P1.

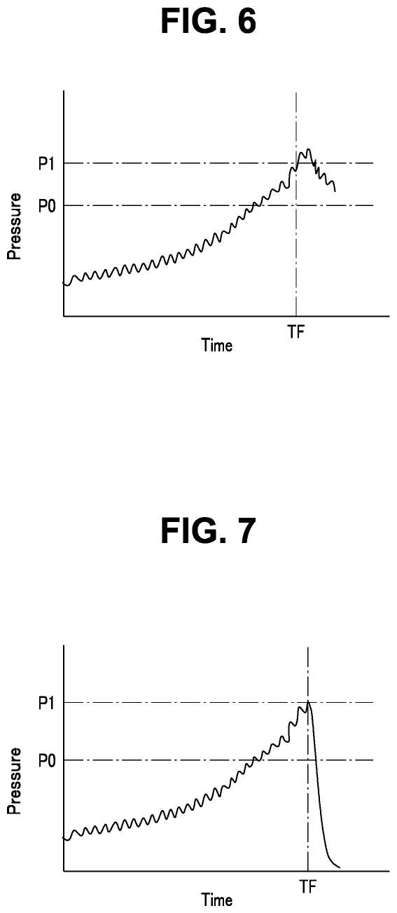

FIG. 6 shows changes in pressure with respect to gas feeding time period in a patient with achalasia according to a conventional pressure control system. In a patient with severe achalasia, even when the pressure reaches, at time TF, the first pressure P1 which is a tolerable pressure at which the examinee feels extremely uncomfortable, the gas within the stomach 100 is not expelled by belching. To alleviate the burden on the examinee, the valve 26 is controlled to close, stopping gas feeding from the gas feeding unit 20. The first pressure P1 is specified as appropriate according to the examinee's conditions (age, gender, and the like), and is, for example, 25 mmHg or higher.

Even when the valve 26 is controlled to close, the gas remaining in the second tube 21 and the gas remaining in the pressure vessel 24 have pressures higher than the internal pressure of the stomach 100, and therefore flow into the stomach 100. For this reason, even when the valve 26 is controlled to close at time TF, the pressure rises to exceed the first pressure P1 and thereafter gradually reduces. That is, the pressure may exceed the first pressure P1, which is the tolerable pressure.

In contrast, in the pressure control apparatus 10, when the pressure reaches (is equal to or greater than) the predetermined first pressure P1 (S18: YES), the process of the controller 15 proceeds to step S19.

### <Step S19>Gas Feeding Stop and First Valve Opening Control

When the pressure reaches the first pressure P1 at time TF, the controller 15 not only controls the valve 26 to close, to thereby stop the gas feeding from the gas feeding unit 20, but also controls the first valve 12 to open. For this reason, the pressure reduces sharply, as shown in FIG. 7. Since there is no risk that the pressure exceeds the first pressure P1 which is a tolerable pressure, the pressure control apparatus 10 can alleviate the burden on the examinee.

It should be noted that when the first valve 12 opens, the internal pressure decreases sharply, thereby causing gastric fluid or the like to flow into the first tube 11 along with the gas, which may contaminate the pressure sensor 14 and the like. For this reason, it may be preferable that the controller 15 perform opening/closing control in which the first valve 12 repeatedly opens and closes, rather than performing control so that the first valve 12 continuously maintains an open state. For example, the controller 15 performs the opening/closing control so that the first valve 12 is open for 0.5 seconds and closed for 0.5 seconds. By performing the opening/closing control, a sharp decrease in internal pressure can be inhibited. The respective time periods of the open and closed time periods are selected as appropriate.

The first filter 13 for reliably inhibiting contamination of the pressure sensor 14 is a disposable component, and may preferably be replaced after each use together with the first tube 11 on a distal end side with respect to the first filter 13.

As described above, the pressure control apparatus 10 according to the present embodiment includes: a first tube 11 communicating with a lumen within a body of an examinee; a pressure sensor 14 disposed on the first tube 11 and configured to measure a pressure of gas; a first valve 12 disposed in the first tube 11 and configured to vent the gas to the atmosphere; and a controller 15 configured to control the first valve 12 to open based on the pressure.

As described above, the pressure control system 1 of the present embodiment includes: an endoscope 9 including a gas/liquid feeding pipe 92 and a forceps channel 91 that pass through an insertion portion 93, the insertion portion 93 being configured to be inserted into a lumen of a subject; a first tube 11 connected to communicate with the forceps channel 91; a pressure sensor 14 disposed on the first tube 11 and configured to measure a pressure of gas; a first valve 12 disposed in the first tube 11 and configured to vent the gas to the atmosphere; and a controller 15 configured to control the first valve 12 to open when the pressure reaches a first pressure P1.

The pressure control method according to the embodiment includes: connecting a first tube 11 to a forceps channel 91 and connecting a second tube 21 to a gas/liquid feeding pipe 92, an endoscope 9 including the gas/liquid feeding pipe 92 and the forceps channel 91 that pass through an insertion portion 93; inserting the insertion portion 93 of the endoscope 9 into a lumen of a subject; supplying gas from a gas supply source to the lumen via the second tube 21 and the gas/liquid feeding pipe 92; measuring a pressure of the gas using a pressure sensor 14 disposed on the first tube 11; and controlling a first valve 12 disposed in the first tube 11 to open when the pressure reaches a first pressure P1, to thereby vent the gas in the lumen to an atmosphere.

According to the present embodiments, a pressure control apparatus that mitigates the burden on the examinee in an examination, a pressure control system including the pressure control apparatus that mitigates the burden on the examinee, and a pressure control method that mitigates the burden on the examinee can be provided.

The above described the pressure control system 1 used for diagnosing GERD and esophageal achalasia, using the stomach 100 as an example of a lumen of the subject. However, the lumen is not limited to the stomach 100, but may be any lumen to which gas is fed, such as, for example, the large intestine.

### Second Embodiment

Pressure control systems 1A and 1B described below are similar to the pressure control system 1 and have substantially the same effects. For this reason, constituent elements with substantially the same functions as those in the pressure control system 1 are designated with the same reference numerals, and descriptions thereof will be omitted.

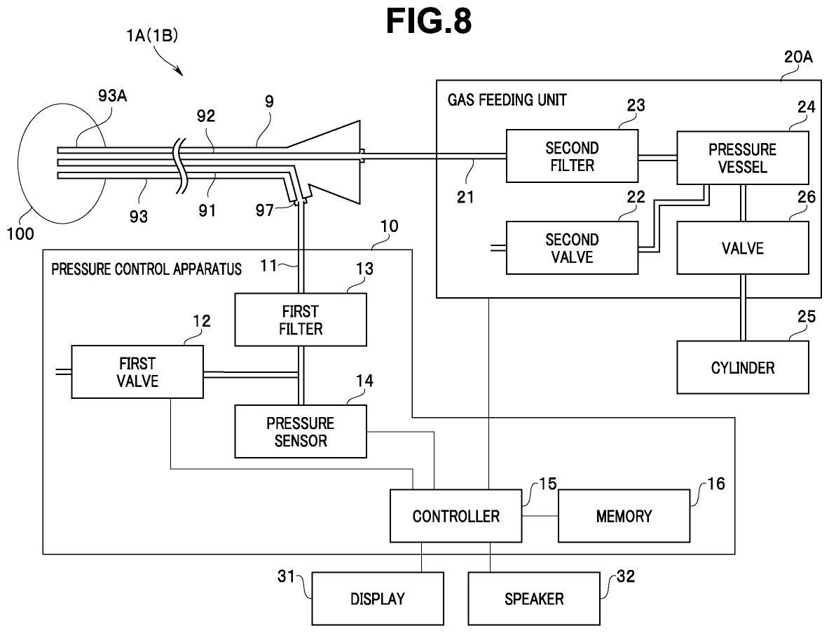

As shown in FIG. 8, a gas feeding unit 20A of the pressure control system 1A of the present embodiment includes a second valve 22 and a second filter 23 disposed in the second tube 21. The second valve 22 and the second filter 23 are hardware. The second valve 22 is a relief valve similar to the first valve 12, and vents gas within the second tube 21 to the atmosphere. The second valve 22 is disposed between the second filter 23 disposed at the second tube 21 and the valve 26, and is disposed, for example, at the pressure vessel 24. The second filter 23 for inhibiting contamination of the pressure vessel 24 and the valve 26 is disposed at a position closer to the stomach 100 than the pressure vessel 24 and the valve 26.

The above-mentioned configuration of the pressure control system 1A is merely one example, and the present disclosure is not limited to the above-mentioned configuration. For example, the second tube 21, in which the second filter 23 and the second valve 22 are disposed, may be attached to the pressure vessel 24 of a general-purpose endoscope system to serve as the gas feeding unit 20A.

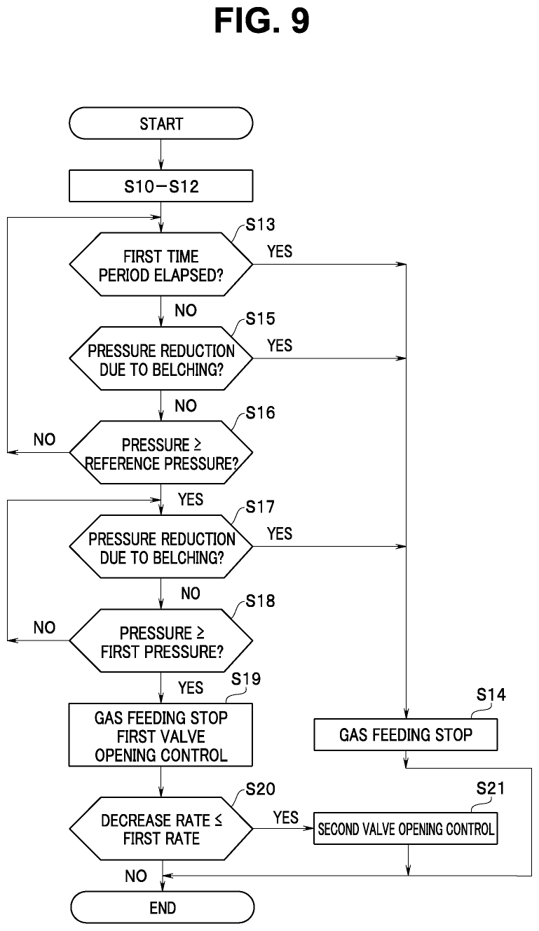

A pressure control method by the pressure control system 1A shown in FIG. 9 is substantially the same as the pressure control method by the pressure control system 1 shown in FIG. 3 from step S10 to step S19, and therefore descriptions thereof will be omitted.

### <Step S20>Decrease Rate≤First Rate?

The controller 15 determines whether a pressure decrease rate ΔP/s is equal to or less than a predetermined first rate ΔP1/s. If the decrease rate ΔP/s is greater than the predetermined first rate ΔP1/s (S20: NO), the controller 15 ends the process. The first rate ΔP1/s is set as appropriate, and is, for example, 5 mmHg/s.

However, if, for example, the first filter 13 is clogged with an unexpectedly large amount of filth, a delay is caused in the emission of gas via the first valve 12. The continuation of a high-pressure state is painful for the examinee. If the decrease rate ΔP/s is equal to or less than the predetermined first rate ΔP1/s (S20: YES), the control of the controller 15 proceeds to step S21.

### <Step S21>Second Valve Opening Control

The controller 15 controls the second valve 22 of the gas feeding unit 20A to open. By venting the gas within the second tube 21 to the atmosphere, the gas within the stomach 100 is also expelled to the atmosphere via the second valve 22. For this reason, the pressure control system 1A can diagnose GERD and achalasia more reliably than the pressure control system 1 without causing discomfort to the examinee.

It should be noted that when the second valve 22 opens, gastric fluid and the like may flow into the second tube 21 along with the gas, contaminating the pressure vessel 24. For this reason, it is preferable that the controller 15 perform opening/closing control in which the second valve 22 repeats open/closed states rather than controlling the second valve 22 to be continuously in an open state. For example, the controller 15 controls the second valve 22 to be in an open state for 0.5 seconds and in a closed state for 0.5 seconds.

There is a possibility that the second filter 23 for reliably inhibiting contamination is contaminated when the second valve 22 opens. For this reason, it may be preferable that the controller 15 generate a signal instructing the user to replace the second filter 23, when controlling the second valve 22 to open. The instruction signal is communicated to the user via the display 31 and a speaker 32.

### Modification of Second Embodiment

Although not shown, the controller 15 of the pressure control system 1B according to the present modification may control the second valve 22 to open instead of the first valve 12 in step S19 of the flowchart shown in FIG. 9. Then in step S21, the controller 15 may control the first valve 12 to open instead of the second valve 22.

That is, the controller 15 may control the second valve 22 to open when the pressure is equal to or greater than the first pressure P1. The controller 15 may control the first valve 12 to open when the pressure decrease rate after controlling the second valve 22 to open is equal to or less than the first rate.

The present disclosure is not limited to the above-mentioned embodiments, etc., and various modifications, combinations, and applications are possible without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), microcontrollers, digital signal processors (DSPs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, ROM, or other storage media) that stores data, software, firmware, or instructions contributing to the processing operations. Accordingly, the processing circuitry may be implemented as a specialized hardware circuit having fixed logic, a programmable circuit executing instructions stored in an internal or external memory, or any combination thereof. The processing circuitry may be configured as, or include, one or more processors. Thus, the term “processor” used in the description of embodiments is to be understood as a specific example of the processing circuitry or as a component included within the processing circuitry. Furthermore, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

In this specification and the appended claims, the singular forms “a,” “an,” and “the” include plural referents unless the context clearly dictates otherwise. For example, reference to “a recess” includes a single recess as well as a plurality of recesses.

Terms of degree, such as “substantially,” “about,” “approximately,” and “generally,” as used herein, represent a reasonable amount of deviation from the specified value or state such that the end result is not significantly changed. These terms should be construed as including a deviation of at least ±5% or ±10% of the modified value, or a deviation recognized by a person of ordinary skill in the art, unless otherwise indicated.

As used herein, geometric, spatial, or directional terms such as “parallel,” “perpendicular,” “orthogonal,” “intersecting,” “polygon,” or specific angles (e.g., “45 degrees”) are not intended to require strict mathematical or geometric exactness. Rather, these terms should be understood to encompass substantial approximations, manufacturing tolerances, and practical variations recognized by those skilled in the art, unless otherwise explicitly claimed.

In the present disclosure, terms such as “same shape,” “uniform shape,” or “common shape” refer to having the same general profile, contour, or topological structure (e.g., both being T-shaped), and do not necessarily require identical dimensions, sizes, scales, or proportions, unless explicitly stated otherwise.

Non-limiting examples according to aspects of the present disclosure will be described in the following clauses:

Clause 1: A pressure control apparatus comprising:
a first tube configured to communicate with a lumen within a body of an examinee;
a pressure sensor disposed on the first tube and configured to measure a pressure of gas;
a first valve disposed in the first tube and configured to vent the gas to an atmosphere;
a second tube configured to communicate with the lumen and supply the gas from a gas supply source to the lumen;
a second valve disposed in the second tube and configured to vent the gas to the atmosphere; and
a controller configured to control the first valve and the second valve to open based on the pressure,
wherein the controller is configured to control the second valve to open when a decrease rate of the pressure after controlling the first valve to open is equal to or less than a first rate.

Clause 2: The pressure control apparatus according to clause 1,
wherein the controller is configured to control the first valve to open when the pressure reaches a first pressure.

Clause 3: The pressure control apparatus according to clause 1,
wherein the controller is configured to control the second valve to open based on a change in the pressure after controlling the first valve to open.

Clause 4: The pressure control apparatus according to clause 1, further comprising:
a first filter positioned closer to the lumen than the first valve of the first tube,
wherein the first filter is a disposable component.

Clause 5: The pressure control apparatus according to clause 1,
wherein the controller is configured to control the second valve to open when the pressure reaches a first pressure.

Clause 6: The pressure control apparatus according to clause 5,
wherein the controller is configured to control the second valve to open based on a change in the pressure after controlling the second valve to open.

Clause 7: The pressure control apparatus according to clause 6,
wherein the controller is configured to control the first valve to open when a decrease rate of the pressure after controlling the second valve to open is equal to or less than a first rate.

Clause 8: The pressure control apparatus according to clause 1,
wherein the first tube and the second tube communicate with respective conduits that pass through an insertion portion of an endoscope.

Clause 9: The pressure control apparatus according to clause 2,
wherein the lumen is a stomach.

Clause 10: A pressure control apparatus comprising:
a first tube configured to communicate with a lumen within a body of an examinee;
a pressure sensor disposed on the first tube and configured to measure a pressure of gas;
a first valve disposed in the first tube and configured to vent the gas to an atmosphere;
a second tube configured to communicate with the lumen and supply the gas from a gas supply source to the lumen;
a second valve disposed in the second tube and configured to vent the gas to the atmosphere; and
a controller configured to control the first valve and the second valve to open based on the pressure,
wherein the controller is configured to:
   control the second valve to open when the pressure reaches a first pressure;
   control the second valve to open based on a change in the pressure after controlling the second valve to open; and
   control the first valve to open when a decrease rate of the pressure after controlling the second valve to open is equal to or less than a first rate.

Clause 11: The pressure control apparatus according to clause 10,
wherein the controller is configured to control the first valve to open when the pressure reaches a first pressure.

Clause 12: The pressure control apparatus according to clause 10,
wherein the controller is configured to control the second valve to open based on a change in the pressure after controlling the first valve to open.

Clause 13: The pressure control apparatus according to clause 10,
wherein the controller is configured to control the second valve to open when a decrease rate of the pressure after controlling the first valve to open is equal to or less than a first rate.

Clause 14: The pressure control apparatus according to clause 10, further comprising:
a first filter positioned closer to the lumen than the first valve of the first tube,
wherein the first filter is a disposable component.

Clause 15: The pressure control apparatus according to clause 10,
wherein the first tube and the second tube communicate with respective conduits that pass through an insertion portion of an endoscope.

Clause 16: The pressure control apparatus according to clause 11,
wherein the lumen is a stomach.

Clause 17: A pressure control system comprising:
an endoscope including a gas/liquid feeding pipe and a forceps channel that pass through an insertion portion, the insertion portion being configured to be inserted into a lumen of an examinee;
a first tube connected to communicate with the forceps channel;
a pressure sensor disposed on the first tube and configured to measure a pressure of gas;
a first valve disposed in the first tube and configured to vent the gas to an atmosphere;
a second tube configured to supply the gas from a gas supply source to the lumen via the gas/liquid feeding pipe;
a second valve disposed in the second tube and configured to vent the gas to the atmosphere; and
a controller configured to:
   control the first valve and the second valve to open when the pressure reaches a first pressure; and
   control the second valve to open when a decrease rate of the pressure after controlling the first valve to open is equal to or less than a first rate.

Clause 18: The pressure control system according to clause 17, further comprising:
a first filter positioned closer to the lumen than the first valve of the first tube,
wherein the first filter is a disposable component.

Clause 19: The pressure control system according to clause 17,
wherein the lumen is a stomach.

What is claimed is:

1. A pressure control system comprising:
a first tube configured to communicate with a lumen within a body of an examinee;
a pressure sensor disposed at the first tube and configured to measure a pressure of a gas;
a first valve disposed at the first tube and configured to vent the gas to an atmosphere;
a second tube configured to communicate with the lumen and supply the gas from a gas supply source to the lumen;
a second valve disposed at the second tube and configured to vent the gas to the atmosphere; and
processing circuitry configured to:
control the first valve to open based on the pressure; and
control the second valve to open when a decrease rate of the pressure after controlling the first valve to open is equal to or less than a predetermined rate.

2. The pressure control system according to claim 1,
wherein the processing circuitry is further configured to control the first valve to open when the pressure is equal to or greater than a first pressure.

3. The pressure control system according to claim 1, further comprising a first filter disposed at the first tube and positioned closer to the lumen than the first valve,
wherein the first filter is a disposable component.

4. The pressure control system according to claim 1,
wherein the first tube and the second tube are further configured to communicate with respective conduits that pass through an insertion portion of an endoscope.

5. The pressure control system according to claim 1,
wherein the lumen is a stomach.

6. The pressure control system according to claim 1, further comprising:
an endoscope comprising:
an insertion portion configured to be inserted into the lumen; and
a gas/liquid feeding pipe and a forceps channel that pass through the insertion portion,
wherein the first tube is further configured to communicate with the forceps channel, and
wherein the second tube is further configured to supply the gas from the gas supply source to the lumen through the gas/liquid feeding pipe.

7. A pressure control system comprising:
a first tube configured to communicate with a lumen within a body of an examinee;
a pressure sensor disposed at the first tube and configured to measure a pressure of a gas;
a first valve disposed at the first tube and configured to vent the gas to an atmosphere;
a second tube configured to communicate with the lumen and supply the gas from a gas supply source to the lumen;
a second valve disposed at the second tube and configured to vent the gas to the atmosphere; and
processing circuitry configured to:
control the second valve to open when the pressure is equal to or greater than a first pressure; and
control the first valve to open when a decrease rate of the pressure after controlling the second valve to open is equal to or less than a predetermined rate.

8. The pressure control system according to claim 7, further comprising a first filter disposed at the first tube and positioned closer to the lumen than the first valve,
wherein the first filter is a disposable component.

9. The pressure control system according to claim 7,
wherein the first tube and the second tube are further configured to communicate with respective conduits that pass through an insertion portion of an endoscope.

10. The pressure control system according to claim 7,
wherein the lumen is a stomach.

11. The pressure control system according to claim 7, further comprising:
an endoscope comprising:
an insertion portion configured to be inserted into the lumen; and
a gas/liquid feeding pipe and a forceps channel that pass through the insertion portion,
wherein the first tube is further configured to communicate with the forceps channel, and
wherein the second tube is further configured to supply the gas from the gas supply source to the lumen via the gas/liquid feeding pipe.

12. A pressure control method implemented by processing circuitry operatively coupled to a pressure control system comprising a first tube, a pressure sensor, a first valve, a second tube, and a second valve, the pressure control method comprising:
controlling the first valve to open based on a pressure of a gas measured by the pressure sensor disposed at the first tube; and
controlling the second valve to open when a decrease rate of the pressure after controlling the first valve to open is equal to or less than a predetermined rate,
wherein the first tube is configured to communicate with a lumen within a body of an examinee,
wherein the first valve is disposed at the first tube and configured to vent the gas to an atmosphere,
wherein the second tube is configured to communicate with the lumen and supply the gas from a gas supply source to the lumen, and
wherein the second valve is disposed at the second tube and configured to vent the gas to the atmosphere.

13. The pressure control method according to claim 12,
wherein controlling the first valve includes opening the first valve when the pressure is equal to or greater than a first pressure.

14. The pressure control method according to claim 12,
wherein the pressure control system further comprises a first filter disposed at the first tube and positioned closer to the lumen than the first valve,
wherein the first filter is a disposable component.

15. The pressure control method according to claim 12,
wherein the first tube and the second tube are further configured to communicate with respective conduits that pass through an insertion portion of an endoscope.

16. The pressure control method according to claim 12,
wherein the lumen is a stomach.

17. The pressure control method according to claim 12,
wherein the pressure control system further comprises an endoscope comprising:
an insertion portion configured to be inserted into the lumen; and
a gas/liquid feeding pipe and a forceps channel that pass through the insertion portion,
wherein the first tube is further configured to communicate with the forceps channel, and
wherein the second tube is further configured to supply the gas from the gas supply source to the lumen through the gas/liquid feeding pipe.
